(19) United States
(12) Patent Application Publication
(10) Pub. No.: US 2018/0235989 A1
(43) Pub. Date: Aug. 23, 2018

(54) TREATMENT AND PREVENTION OF NEISSERIA GONORRHOEAE INFECTION USING CMP-ACTIVATED NONULOSONATE ANALOG COMPOUNDS

(71) Applicant: NATIONAL RESEARCH COUNCIL CANADA, OTTAWA (CA)

(72) Inventor: IAN C. SCHOENHOFEN, STITTSVILLE (CA)

(21) Appl. No.: 15/753,345

(22) PCT Filed: Aug. 19, 2016
(86) PCT No.: PCT/CA2016/050977

§ 371 Date: Feb. 19, 2018

Related U.S. Application Data
(60) Provisional application No. 62/207,246, filed on Aug. 19, 2015.

(51) Int. Cl. A61K 31/7012; A61K 45/06; A61K 31/7068

(52) U.S. Cl. CPC A61K 31/7012; A61K 31/7068; A61K 45/06

(57) ABSTRACT

There is provided a method for treating or preventing a Neisseria gonorrhoeae infection in a subject, comprising administering to the subject an effective amount of a compound of general formula I below or a pharmaceutical composition comprising said compound, or a derivative thereof, or a pharmaceutically acceptable salt thereof, or a solvate or hydrate thereof, or a stereoisomer thereof.

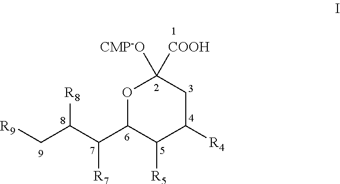

### FIELD OF THE INVENTION

The present invention relates to medical conditions involving Neisseria gonorrhoeae. More specifically, the present invention relates to methods of treating and preventing Neisseria gonorrhoeae infection that are based on cytidine 5′-monophospho-nonulosonate (CMP-NulO) analog compounds.

### BACKGROUND OF THE INVENTION

Sialic acids are a family of 9 carbon sugars (belonging to a larger family of nonoses, or nonulosonates) expressed in the tissues of every vertebrate and several “higher-order” invertebrates [1]. Sialic acids serve a wide variety of biological roles, including modulating several aspects of immune function [2]. For example, cell surface-associated sialic acid inhibits complement activation. As an example of immune regulation, sheep erythrocytes are resistant to lysis by the alternative pathway because surface sialic acids increase the affinity of factor H (fH; inhibitor of the alternative pathway) [3]. Neuraminidase treatment of sheep erythrocytes then reduces the affinity of fH, which permits complement activation and promotes hemolysis. Recent work showed that fH C-terminal domains 19 and 20 bound simultaneously to C3b (complement factor that binds microbial cell surfaces) and glycosaminoglycans (including sialic acids), respectively, on host cells, which served to inhibit the alternative pathway [4]. Loss of sialic acids decreased fH binding and enhanced activation of the alternative pathway. Typically, fH binds vertebrate cell surfaces via sialic acids to allow preferential protection of host cells (i.e. reduce complement-mediated damage).

Many microbes express sialic acids, as well as other unique microbial nonulosonates (i.e. legionaminic (Leg) and pseudaminic (Pse) acid), on their surfaces that contribute to pathogenesis in several ways including subversion of complement activation, promoting biofilm formation and facilitating colonization [5]. Some pathogens such as Neisseria gonorrhoeae, Haemophilus influenzae, Histophilus somni (Haemophilus somnus) and group A N. meningitidis lack the ability to synthesize sialic or nonulosonic acids, but scavenge these molecules (such as Neu5Ac or Neu5Gc, or the CMP-activated form CMP-Neu5Ac) from the host. Other pathogens, for example, Escherichia coli K1, Streptococcus agalactiae, groups B, C, W, and Y N. meningitidis, Campylobacter jejuni and certain Leptospira, can synthesize nonulosonic acids such as Neu5Ac, Leg5Ac7Ac or Pse5Ac7Ac de novo. Sialylation of gonococcal lacto-N-neotetraose (LNnT) lipooligosaccharide (LOS) enhances resistance of N. gonorrhoeae to complement-dependent killing by decreasing binding of IgG against select bacterial targets such as the porin B (PorB) protein [6], which attenuates the classical pathway. LNnT LOS sialylation also enhances fH binding, which results in inhibition of the alternative pathway [7].

N. gonorrhoeae has become resistant to almost every conventional antibiotic. Over the past 3 years, resistance to ceftriaxone has ushered in an era of potentially untreatable gonorrhea. There is an urgent need for novel therapeutics and vaccines against this disease. LOS sialylation is an important aspect of gonococcal pathogenesis and isogenic mutants that lack the ability to sialylate their LOS are at a disadvantage in vivo compared to their wild-type counterparts [8]. Disabling the ability of gonococci to sialylate their LOS represents a novel prophylactic or treatment strategy.

U.S. patent application Ser. No. 14/627,396 discloses cytidine 5′-monophospho-nonulosonate (CMP-NulO) analog compounds for treating or preventing Neisseria gonorrhoeae infection in a subject.

Also, the inventor is aware of these other documents [36-46].

There is a need for CMP-NulO analog compounds that provide a more efficient treatment or prevention. Also, there is a need for CMP-NulO analog compounds that present low toxicity effects in a subject.

### SUMMARY OF THE INVENTION

The invention is drawn to a method of treating or preventing Neisseria gonorrhoeae infection in a subject that is based on cytidine 5′-monophospho-nonulosonate (CMP-NulO) analog compounds. More specifically, the compounds of the invention relate to CMP-3-deoxy-D-glycero-D-galacto-nonulosonic acid (CMP-KDN). Since 3-deoxy-D-glycero-D-galacto-nonulosonic acid (KDN, also called 3-deoxy-D-glycero-D-galacto-2-nonulosonic acid or 2-keto-3-deoxy-D-glycero-D-galacto-nononic acid) is a sugar found in humans at low levels, it is anticipated that any toxic effects associated to the use of the compounds of the invention will be low.

The invention thus provides the following according to aspects thereof:
(1). A method for treating or preventing a Neisseria gonorrhoeae infection in a subject, comprising administering to the subject an effective amount of a compound of general formula I below or a pharmaceutical composition comprising said compound, or a derivative thereof, or a pharmaceutically acceptable salt thereof, or a solvate or hydrate thereof, or a stereoisomer thereof

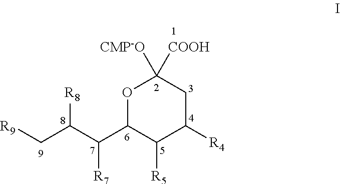

wherein:
R₅ is selected from the group consisting of: XR wherein X is O or S and R is H or a C₁ to C₆ linear, branched, saturated or unsaturated alkyl or cycloalkyl; NR′R″ wherein R′ and R″ are each independently H, a C₁ to C₆ linear, branched, saturated or unsaturated alkyl or cycloalkyl, or a substituted or unsubstituted phenyl or alkyl phenyl, or R′ and R″ together with N form a 5- or 6-member ring, optionally the ring is substituted with a C₁ to C₃ alkyl; XCYR wherein X and Y are each independently O or S and R is a C₁ to C₆ linear, branched, saturated or unsaturated alkyl or cycloalkyl or R is a substituted or unsubstituted phenyl or alkyl phenyl; and a halogen atom which is F, Cl, Br or I; and
R₄ and R₇ to R₉ are each independently selected from the group consisting of: H; XR wherein X is O or S and R is H or a C₁ to C₆ linear, branched, saturated or unsaturated alkyl or cycloalkyl; OR′R″ wherein R′ and R″ are each independently H or a C₁ to C₆ linear, branched, saturated or unsaturated alkyl or cycloalkyl; XCYR wherein X and Y are each independently O or S and R is a C₁ to C₆ linear, branched, saturated or unsaturated alkyl or cycloalkyl or R is a substituted or unsubstituted phenyl or alkyl phenyl; NR′R″ wherein R′ and R″ are each independently H, a C₁ to C₆ linear, branched, saturated or unsaturated alkyl or cycloalkyl, or R′ and R″ together with N form a 5- or 6-member ring, optionally the ring is substituted with a C₁ to C₃ alkyl; NH-acetyl; NH-thio-acetyl; NH-azido-acetyl; NH-(D-alanyl); NH-(N-acetyl-D-alanyl); N₃; O-Sia; O-Glc; benzamido [NHCOPh]; NH-Gly; NH-Succ; hexanoylamido [NHCO(CH₂)₄CH₃]; O-lactyl; O-phosphate; O-sulfate; and a halogen atom which is F, Cl, Br or I.
(2). The method of (1), wherein:
   R₄ is OH, O-acetyl, O-methyl, or NH₂;
   R₅ is OH, O-acetyl, O-methyl, or sulfhydryl;
   R₇ is OH, NH₂, O-acetyl, O-methyl, NH-acetyl, NH-azido-acetyl, NH-(D-alanyl), NH-(N-acetyl-D-alanyl), F, H, or N₃;
   R₅ is OH, NH₂, N₃, O-acetyl, O-methyl, O-sulfate, O-Sia, or O-Glc; and
   R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.
(3). The method of (1), wherein the compound is of general formula IA below

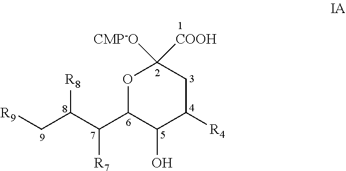

wherein R₄ and R₇ to R₉ are each independently as defined in (1).
(4). The method of (1), wherein the compound is of general formula II below

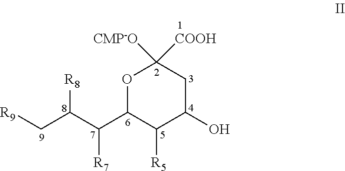

wherein R₅ and R₇ to R₉ are each independently as defined in (1).
(5). The method of (1), wherein the compound is of general formula II below

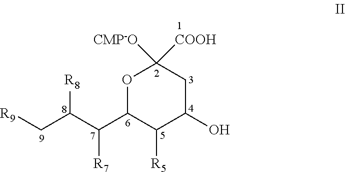

wherein:
R₅ is OH, O-acetyl, O-methyl, or sulfhydryl;
R₇ is OH, NH₂, O-acetyl, O-methyl, NH-acetyl, NH-azido-acetyl, NH-(D-alanyl), NH-(N-acetyl-D-alanyl), F, H, or N3;
R₅ is OH, NH₂, N₃, O-acetyl, O-methyl, O-sulfate, O-Sia, or O-Glc; and
R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.
(6). The method of (1), wherein the compound is of general formula II below

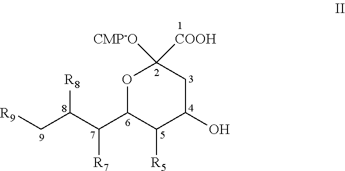

wherein:
R₅ is OH, F, Cl, Br, methyl, O-acetyl, O-methyl, or sulfhydryl;
R₇ OH, NH₂, O-acetyl, O-methyl, NH-acetyl, NH-azido-acetyl, NH-(D-alanyl), NH-(N-acetyl-D-alanyl), F, H, or N₃;
R₅ OH, NH₂, N₃, O-acetyl, O-methyl, O-sulfate, O-Sia, or O-Glc; and
R₉ OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, 0-lactyl, O-phosphate, O-sulfate, O-Sia, F or H.
(7). The method of (1), wherein the compound is of general formula IIA below

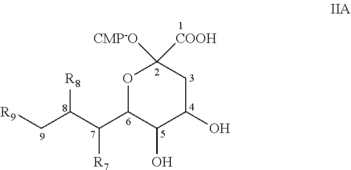

wherein R₇ to R₉ are each independently as defined in (1).
(8). The method of (1), wherein the compound is of general formula IIA below

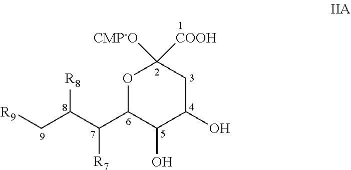

wherein:
R₇ is OH, NH₂, O-acetyl, O-methyl, NH-acetyl, NH-azido-acetyl, NH-(D-alanyl), NH-(N-acetyl-D-alanyl), F, H, or N₃;
R₅ is OH, NH₂, N₃, O-acetyl, O-methyl, O-sulfate, O-Sia, or O-Glc; and
R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.
(9). The method of (1) wherein the compound is of general formula III below

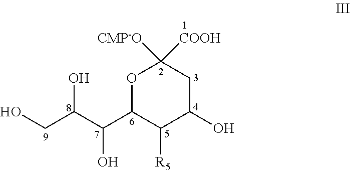

wherein R₅ is as defined in (1).
(10). The method of (1), wherein the compound is of general formula IV below

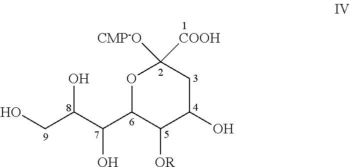

wherein R is H or a C₁ to C₆ linear, branched, saturated or unsaturated alkyl or cycloalkyl.
(11). The method of (1), wherein the compound is compound V below

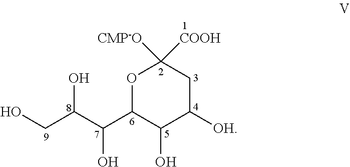

(12). The method of (1), wherein the compound is cytidine 5′-monophospho-3-deoxy-D-glycero-D-galacto-nonulosonic acid (CMP-KDN) below

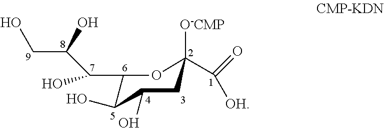

(13). The method of (1), wherein the compound is of general formula VI below

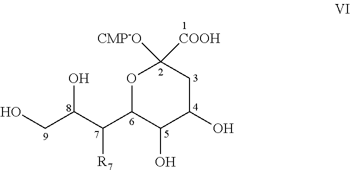

wherein R₇ is as defined in (1).
(14). The method of (1), wherein the compound is of general formula VI below

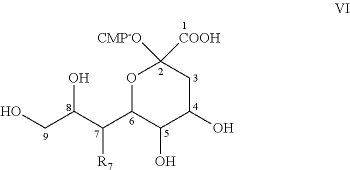

wherein R₇ is N₃, O-methyl, O-acetyl, NH₂ or a halogen atom.
(15). The method of (1), wherein the compound is compound VII below

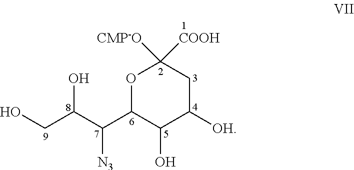

(16). The method of (1), wherein the compound is cytidine 5′-monophospho-3,7-dideoxy-D-glycero-D-galacto-nonulosonic acid (CMP-KDN7N₃) below

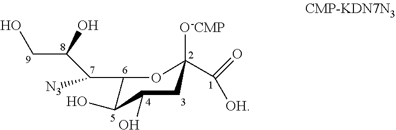

(17). The method of (1), wherein the compound is:
   3-deoxy-9-O-acetyl-D-glycero-D-galacto-nonulosonic acid (KDN9OAc) (R₄═OH, R₅═OH, R₇═OH, R₈═OH, R₉═O-acetyl);
   3-deoxy-8-O-acetyl-D-glycero-D-galacto-nonulosonic acid (KDN8OAc) (R₄═OH, R₅═OH, R₇═OH, R₈═O-acetyl, R₉═OH);
   3-deoxy-7-O-acetyl-D-glycero-D-galacto-nonulosonic acid (KDN7OAc) (R₄═OH, R₅═OH, R₇═O-acetyl, R₈═OH, R₉═OH);
   3-deoxy-5-O-acetyl-D-glycero-D-galacto-nonulosonic acid (KDN5OAc) (R₄═OH, R₅═O-acetyl, R₇═OH, R₈═OH, R₉═OH);
   3-deoxy-4-O-acetyl-D-glycero-D-galacto-nonulosonic acid (KDN4OAc) (R₄═O-acetyl, R₅═OH, R₇═OH, R₈═OH, R₉═OH);
   3-deoxy-8,9-di-O-acetyl-D-glycero-D-galacto-nonulosonic acid (KDN8,9diOAc) (R₄═OH, R₅═OH, R₇═OH, R₈═O-acetyl, R₉═O-acetyl);
   3-deoxy-9-O-methyl-D-glycero-D-galacto-nonulosonic acid (KDN9OMe) (R₄═OH, R₅═OH, R₇═OH, R₈═OH, R₉═O-methyl);
   3-deoxy-8-O-methyl-D-glycero-D-galacto-nonulosonic acid (KDN8OMe) (R₄═OH, R₅═OH, R₇═OH, R₈═O-methyl, R₉═OH);
   3-deoxy-7-O-methyl-D-glycero-D-galacto-nonulosonic acid (KDN7OMe) (R₄═OH, R₅═OH, R₇═O-methyl, R₈═OH, R₉═OH);
   3-deoxy-5-O-methyl-D-glycero-D-galacto-nonulosonic acid (KDN5OMe) (R₄═OH, R₅═O-methyl, R₇═OH, R₈═OH, R₉═OH);
   3-deoxy-4-O-methyl-D-glycero-D-galacto-nonulosonic acid (KDN4OMe) (R₄═O-methyl, R₅═OH, R₇═OH, R₈═OH, R₉═OH);
   3-deoxy-8,9-di-O-methyl-D-glycero-D-galacto-nonulosonic acid (KDN8,9diOMe) (R₄═OH, R₅═OH, R₇═OH, R₈═O-methyl, R₉═O-methyl);
   3,9-dideoxy-D-glycero-D-galacto-nonulosonic acid (9-deoxy-KDN) (R₄═OH, R₅═OH, R₇═OH, R₅═OH, R₉═H);
   3,7-dideoxy-D-glycero-D-galacto-nonulosonic acid (7-deoxy-KDN) (R₄═OH, R₅═OH, R₇═H, ═OH, R₈═OH);
   3,9-dideoxy-9-azido-D-glycero-D-galacto-nonulosonic acid (KDN9Az) (R₄═OH, R₅═OH, R₇═OH, R₅═OH, R₉═N₃);
   3,9-dideoxy-9-fluoro-D-glycero-D-galacto-nonulosonic acid (KDN9F) (R₄═OH, R₅═OH, R₇═OH, R₅═OH, R₉═F); or
   3,7-dideoxy-7-fluoro-D-glycero-D-galacto-nonulosonic acid (KDN7F) (R₄═OH, R₅═OH, R₇═F, R₈═OH, R₉═OH).
(18). The method of any one of (1) to (17), wherein the pharmaceutical composition comprises the compound and a pharmaceutically acceptable carrier.
(19). The method of any one of (1) to (18), wherein the pharmaceutical composition comprises the compound and another therapeutic compound.
(20). The method of any one of (1) to (18), wherein the pharmaceutical composition comprises the compound and another therapeutic compound selected from the group consisting of: other compounds used in the treatment or prevention of Neisseria gonorrhoeae infection, compounds used in the treatment or prevention of sexually transmitted diseases including Chlamidia trachomatis infection and HIV, and antibacterial peptides.
(21). The method of any one of (1) to (20), wherein the subject is a mammal.
(22). The method of any one of (1) to (20), wherein the subject is a human.
(23). Use, for treating or preventing a Neisseria gonorrhoeae infection in a subject, of a compound as defined in any one of (1) to (17).
(24). Use, for treating or preventing a Neisseria gonorrhoeae infection in a subject, of a pharmaceutical composition as defined in any one of (1) to (17).
(25). Use, in the preparation of a medicament for treating or preventing a Neisseria gonorrhoeae infection in a subject, of a compound as defined in any one of (1) to (17).
(26). A device coated or filled with a compound as defined in any one of (1) to (17).
(27). A device coated or filled with a pharmaceutical composition as defined in any one of (1) to (17).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

Example embodiments are illustrated by way of example and not limitation in the figures of the accompanying drawings, in which:

FIG. 1: CMP-KDN serves as a substrate for gonococcal LOS and incorporation of KDN on gonococcal lacto-N-neotetraose LOS enhances factor H (FH) binding. A. Incorporation of KDN by gonococci. Gonococcal strain F62 ΔIgtD (expresses lacto-N-neotetraose (LNnT) from HepI) was grown in media containing CMP-KDN (20 μg/mL) and binding of mAb 3F11 was measured by flow cytometry. mAb 3F11 binds only to sialylated LNnT; substitution of the terminal Gal of LNnT, for example with a NulO, will decrease mAb 3F11 binding. Approximately 5×10⁷ bacteria were incubated with mAb 3F11 tissue culture supernatants for 15 minutes at 37° C. Binding of mAb 3F11 was disclosed with anti-mouse IgM FITC (Sigma; dilution of 1:100). Bacteria grown in CMP-Neu5Ac or in media alone were used as positive and negative controls for sialylation, respectively. A representative histogram is shown in the upper panel. The bar graph shows the average of the median fluorescence from two independent observations. B. Factor H (FH) binding to gonococci grown in CMP-KDN. Approximately 5×10⁷ bacteria grown in media alone (no sialic acid), media plus CMP-Neu5Ac (20 μg/mL; positive control for FH binding) or in media containing CMP-KDN (20 μg/mL) were incubated with purified human FH (20 μg/mL) in HBSS' for 15 minutes at 37° C. and bound FH was detected with affinity isolated goat anti-human FH followed by anti-goat IgG FITC (Sigma; 1:100 dilution). A representative histogram is shown in the upper panel. The bar graph shows the average of the median fluorescence from two independent observations.

FIG. 2: Female BALB/c mice 5-6 weeks of age (Jackson Laboratories) in the diestrus phase of the estrous cycle were started on treatment (that day) with 0.5 mg of Premarin (Pfizer) given subcutaneously on each of three days; −2, 0 and +2 days (before, the day of and after inoculation) to prolong the estrus phase of the cycle and promote susceptibility to N. gonorrhoeae infection. Antibiotics (vancomycin, colistin, neomycin, trimethoprim and streptomycin) that were ineffective against N. gonorrhoeae were also used to reduce competitive microflora. Mice (n=40) were then infected with 10⁶ CFU of strain H041. Three groups of mice (n=10/group) were treated with 10 μg intravaginally daily (first dose was administered 30 minutes before the introduction of bacteria) with one of the following CMP-NulOs in normal saline: CMP-Leg5Ac7Ac, CMP-Neu5Ac9Az or CMP-KDN. A fourth group (n=10) was given saline (vehicle control). Vaginal swabs were obtained daily from each animal, serially diluted and plated on chocolate agar containing vancomycin, colistin, neomycin, trimethoprim and streptomycin (VCNTS) to quantify bacterial loads. A. Median time to clearance was estimated using Kaplan-Meier survival curves; the times to clearance were compared between groups using a log-rank test. P<0.0001 for the control versus each of the treatment groups. Time to clearance between each of the treatment groups was similar (P>0.05). B. The mean area under the curve (log₁₀CFU vs. time) was computed for each mouse to estimate the bacterial burden over time (cumulative infection); the means under the curves were compared between groups using the Kruskal-Wallis nonparametric rank sum test because distributions were skewed or kurtotic. Groups were compared using Dunn's multiple comparison test. ****, P<0.0001; **, P<0.01; *, P<0.05. The differences among the groups that received CMP-NulOs were not significant.

FIG. 3: CMP-KDN7N₃ serves as a substrate for gonococcal Lst resulting in incorporation of KDN7N₃ on gonococcal lacto-N-neotetraose (LNnT) LOS—Incorporation of KDN7N₃ by gonococci. Gonococcal strain F62ΔIgtD (expresses lacto-N-neotetraose (LNnT) from HepI) was grown in media containing CMP-KDN7N₃ (20 μg/mL) and binding of mAb 3F11 was measured by flow cytometry. mAb 3F11 binds only to sialylated LNnT; substitution of the terminal Gal of LNnT, for example with a NulO, will decrease mAb 3F11 binding. Approximately 5×10⁷ bacteria were incubated with mAb 3F11 tissue culture supernatants for 15 minutes at 37° C. Binding of mAb 3F11 was disclosed with anti-mouse IgM FITC (Sigma; dilution of 1:100). Bacteria grown in CMP-Neu5Ac or in media alone were used as positive and negative controls for sialylation, respectively. A representative histogram is shown in the upper panel (A), and a bar graph below shows the median fluorescence for each condition (B).

Other features of the present embodiments will be apparent from the accompanying drawings and from the detailed description that follows.

### DESCRIPTION OF ILLUSTRATIVE EMBODIMENTS

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this disclosure pertains.

The use of the word “a” or “an” when used in conjunction with the term “comprising” in the claims and/or the description may mean “one”, but it is also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “effective amount” is an amount of the CMP-nonulosonate analog compound that is sufficient to treat a N. gonorrhoeae infection, that is, to accomplish at least one of the following: reduce virulence of N. gonorrhoeae, reduce the rate of transmission of N. gonorrhoeae, and reduce the severity of one or more symptoms associated with N. gonorrhoeae infection, for example, burning sensation during urination, painful or swollen testicles and increased vaginal discharge.

As used herein, the term “subject” is understood as being any mammal including a human being treated with a compound of the invention.

As used herein, the term “derivative” is understood as being a substance which comprises the same basic carbon skeleton and carbon functionality in its structure as a given compound, but can also bear one or more substituents or rings.

As used herein, the term “analog” is understood as being a substance similar in structure to another compound but differing in some slight structural detail.

As used herein, the term “salt” is understood as being acidic and/or basic salts formed with inorganic and/or organic acids or bases. Zwitterions (internal or inner salts) are understood as being included within the term “salt” as used herein, as are quaternary ammonium salts such as alkylammonium salts. Nontoxic, pharmaceutically acceptable salts are preferred, although other salts may be useful, as for example in isolation or purification steps.

Examples of acid addition salts include but are not limited to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, phosphoric, 2-hydroxyethanesulfonate, lactate, maleate, mandelate, methanesulfonate, 2-naphthalenesulfonate, nicotinate, oxalate, pectinate, persulfate, 3-phenylpropionate, picrate, pivalate, propionate, succinate, tartrate, thiocyanate, tosylate, and undecanoate.

Salts may also be made from inorganic acids such as hydrochloric, hydrobromic, sulfuric, sulfamic, phosphoric and nitric acids.

Examples of base addition salts include but are not limited to alkali metal salts and alkaline earth metal salts. Non limiting examples of alkali metal salts include lithium, sodium and potassium salts. Non-limiting examples of alkaline earth metal salts include magnesium and calcium salts.

As used herein the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “a therapeutically effective amount”. Effective amounts for each purpose will depend on the severity of the disease or injury as well as the weight and general state of the subject.

As used herein the terms “treatment” and “treating” mean the management and care of a subject for the purpose of combating a condition, such as a disease or disorder. The term is intended to include the full spectrum of treatments for a given condition from which the patient is suffering, such administration of the active compounds to alleviate the symptoms or complications, to delay the progression of the condition, and/or to cure or eliminate the condition. The subject to be treated is preferably a mammal, in particular a human being.

The present disclosure is drawn to a method of treating or preventing Neisseria gonorrhoeae infection in a subject that is based on cytidine 5′-monophospho-nonulosonate (CMP-NulO) analog compounds. More specifically, the compounds of the invention relate to CMP-3-deoxy-D-glycero-D-galacto-nonulosonic acid (CMP-KDN). Since 3-deoxy-D-glycero-D-galacto-nonulosonic acid (KDN, also called 3-deoxy-D-glycero-D-galacto-2-nonulosonic acid or 2-keto-3-deoxy-D-glycero-D-galacto-nononic acid) is a sugar found in humans at low levels, it is anticipated that any toxic effects associated to the use of the compounds of the invention will be low.

Indeed, 3-deoxy-D-glycero-D-galacto-nonulosonic acid (KDN) is a sialic acid or nonulosonate (NulO) that is ubiquitously expressed in vertebrates during normal development and tumorigenesis. In KDN, the N-acetyl group at C5 of N-acetyl-neuraminic acid is replaced by a hydroxyl group, and again is found in vertebrate glycoconjugates and bacterial polysaccharides, where it was first identified in rainbow trout egg polysialoglycoprotein in 1986 [9,10]. Its expression is thought to involve i) mannose-6-phosphate+phosphoenolpyruvate (PEP)→KDN-9-phosphate (KDN-9-P)+Pi; ii) KDN-9-P→KDN+Pi; iii) KDN+CTP→CMP-KDN+PPi; and iv) CMP-KDN+R-OH→R—O-KDN+CMP (R, acceptor glycan) [9,10]. In summary, KDN occurs widely among vertebrates and bacteria, is found in almost all types of glycoconjugates, can be linked to almost all glycan structures in place of Neu5Ac, and its biosynthesis involves mannose, CMP-activation of KDN and transfer to acceptor sugar residues [10].

Using crude enzyme preparations, it has been shown that mammalian CMP-sialic acid synthetases (enzymes responsible for step iii) above) have very low activity/ability to synthesize CMP-KDN from KDN and CTP, relative to enzymes from rainbow trout [11,12]. In humans, the Neu5Ac-9-phosphate synthase (step i) above) can catalyze the synthesis of both Neu5Ac-9-phosphate and KDN-9-phosphate from aldol condensation of PEP with substrates ManNAc-6-phosphate or Man-6-phosphate, respectively [13-15]. In addition, the human CMP-sialic acid synthetase can CMP-activate KDN [14]. Importantly, although a minor component, KDN has been reported to be present in human tissues [16-19] and therefore is likely to be poorly immunogenic. Moreover, KDN in the context of glycoconjugates has been demonstrated to be sialidase resistant [20,21]. Due to the inherent presence of KDN in human tissues, its sialidase resistance (i.e., stability) and its relative ease of chemical synthesis [22,23], CMP-KDN is an attractive therapeutic agent for humans.

The compounds of the invention are of general formula I, IA, II, IIA, III, IV, V, VI, VII as outlined below.

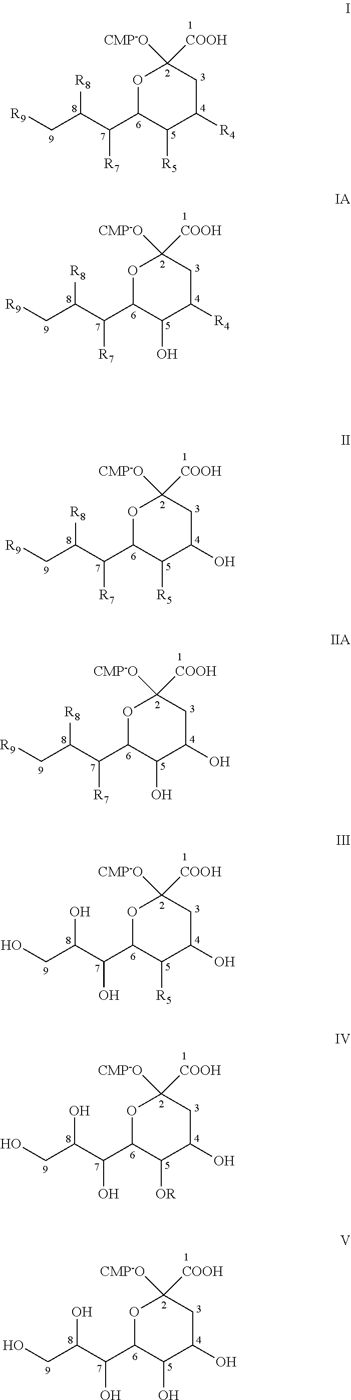

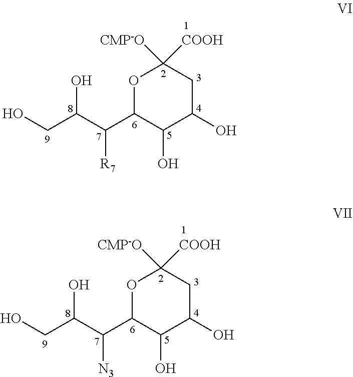

wherein:
R₅ is selected from the group consisting of: XR wherein X is O or S and R is H or a C₁ to C₆ linear, branched, saturated or unsaturated alkyl or cycloalkyl; NR′R″ wherein R′ and R″ are each independently H, a C₁ to C₆ linear, branched, saturated or unsaturated alkyl or cycloalkyl, or a substituted or unsubstituted phenyl or alkyl phenyl, or R′ and R″ together with N form a 5- or 6-member ring, optionally the ring is substituted with a C₁ to C₃ alkyl; XCYR wherein X and Y are each independently O or S and R is a C₁ to C₆ linear, branched, saturated or unsaturated alkyl or cycloalkyl or R is a substituted or unsubstituted phenyl or alkyl phenyl; and a halogen atom which is F, Cl, Br or I; and
R₄ and R₇ to R₉ are each independently selected from the group consisting of: H; XR wherein X is O or S and R is H or a C₁ to C₆ linear, branched, saturated or unsaturated alkyl or cycloalkyl; OR′R″ wherein R′ and R″ are each independently H or a C₁ to C₆ linear, branched, saturated or unsaturated alkyl or cycloalkyl; XCYR wherein X and Y are each independently O or S and R is a C₁ to C₆ linear, branched, saturated or unsaturated alkyl or cycloalkyl or R is a substituted or unsubstituted phenyl or alkyl phenyl; NR′R″ wherein R′ and R″ are each independently H, a C₁ to C₆ linear, branched, saturated or unsaturated alkyl or cycloalkyl, or R′ and R″ together with N form a 5- or 6-member ring, optionally the ring is substituted with a C₁ to C₃ alkyl; NH-acetyl; NH-thio-acetyl; NH-azido-acetyl; NH-(D-alanyl); NH-(N-acetyl-D-alanyl); N₃; O-Sia; O-Glc; benzamido [NHCOPh]; NH-Gly; NH-Succ; hexanoylamido [NHCO(CH₂)₄CH₃]; O-lactyl; O-phosphate; O-sulfate; and a halogen atom which is F, Cl, Br or I.

Also, compounds of the invention include compound CMP-KDN and compound CMP-KDN7N₃ outlined below.

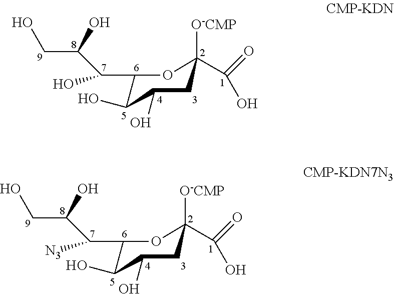

Moreover, compounds of the invention include the following:

In some embodiments, the formulation may include or may further comprise enzymatic inhibitors, pH modulating compounds, buffers, salt formation, solubilizers, excipients, emulsifiers, surfactants and/or antioxidants or the like. Such pharmaceutical compositions are also envisioned and are within the scope of the invention. For example, the formulation may include a sialyltransferase, for example, Lst or another suitable sialyltransferase in the formulation.

In other embodiments, the formulation may be a sustained release formulation. The term “sustained release” as used herein refers to the release of a drug or compound at a predetermined rate in order to maintain a specific concentration for a specific period of time. Sustained release formulations are well known in the art and may comprise for example a hydrogel, liposomes or a polymer.

In some embodiments, the formulation may include or may further comprise enzymatic inhibitors, pH modulating compounds, buffers, salt formation, solubilizers, excipients, emulsifiers, surfactants and/or antioxidants or the like. Such pharmaceutical compositions are also envisioned and are within the scope of the invention. For example, the formulation may include a sialyltransferase, for example, Lst or another suitable sialyltransferase in the formulation.

Suitable products will be readily apparent to one of skill in the art. For example, one or more of the CMP-nonulosonate sugars of the invention may be formulated for intravenous or topical administration, as discussed herein.

For oral administration, the CMP-nonulosonate analog compounds may be formulated in a tablet, coated tablet, capsule or other similar form known in the art for oral administration of medicaments.

For topical administration, the CMP-nonulosonate analog compounds may be formulated in a spray, cream, lotion, ointment or similar product, as well as a device similar to that used for yeast infections (i.e., including tablet or the like). This device could be used for treatment and prophylaxis. In other embodiments, the CMP-nonulosonate analog compounds may be formulated for release from a prophylactic device. Examples of suitable prophylactic devices include but are by no means limited to condoms, cervical caps, contraceptive diaphragms, vaginal rings, devices used for yeast infections and the like.

As will be appreciated by one of skill in the art, in some embodiments of the invention, the formulation comprises an effective amount of one or more of the cytidine 5′-monophospho-nonulosonate analog compounds and is used as a treatment for a subject who has or is suspected of having or is at risk of a N. gonorrhoeae infection. For example, the cytidine 5′-monophospho-nonulosonate sugars may be incorporated into a formulation as disclosed herein and formulated as a medicament for treatment of a N. gonorrhoeae infection. For example, the medicament comprising the cytidine 5′-monophospho-nonulosonate sugars may be formulated for oral, intravenous administration or for topical administration. As discussed herein, the medicament may also be formulated for sustained release.

In other embodiments of the invention, there is provided a prophylactic device coated or filled with an effective amount of one or more of the cytidine 5′-monophospho-nonulosonate analog compounds defined above for treating an individual who has or is suspected of having or is at risk of an N. gonorrhoeae infection. In some embodiments, the cytidine 5′-monophospho-nonulosonate analog compound is formulated for sustained release, as discussed above.

As will be appreciated by one of skill in the art, a subject who is “at risk” of a N. gonorrhoeae infection is a subject who may have sexual contact with another subject who may be infected by N. gonorrhoeae.

### DESCRIPTION OF A PREFERRED EMBODIMENT

### Synthesis of CMP-3-Deoxy-D-Glycero-D-Galacto-Nonulosonic Acid or CMP-Deaminated Neuraminic Acid (CMP-KDN)

KDN (3-deoxy-D-glycero-D-galacto-nonulosonic acid) was enzymatically prepared using a Pasteurella multocida aldolase [24]. Typically, reactions contained 100 mM Tris pH 7.5, 20 mM mannose, 100 mM sodium pyruvate, and approximately 0.15 mg/mL aldolase. Reactions were incubated at 37° C. with gentle shaking for 24-48 hours, and finally enzyme was removed by centrifugal ultrafiltration. Next, CMP-activation of synthesized KDN was achieved enzymatically using a CMP-sialic acid synthetase from Campylobacter jejuni [25]. Here, reactions typically contained 50 mM Tris pH 8.5, 50 mM MgCl₂, 5 mM CTP, approximately 5 mM KDN, 4 units pyrophosphatase per mmole of CTP and approximately 0.1 mg/mL of CMP-sialic acid synthetase. Reactions were incubated at 37° C. for 2 hours, and finally enzyme was removed by centrifugal ultrafiltration. The filtered CMP-KDN was then purified using a Q sepharose fast flow (GE Healthcare) column equilibrated in 1 mM NaCl. Before sample application, the CMP-KDN preparation was diluted approximately 40 times in 1 mM NaCl. After sample application, the resin was washed with 2 CV of 1 mM NaCl and purified CMP-KDN was obtained with a 0.8 CV 100 mM NaCl step elution. This CMP-KDN preparation was further desalted using diafiltration, where the sample was transferred to a diafiltration cell (Diaflo ultrafiltration membranes, YCOS 76 mm), and filtered using 3 times the volume of 1 mM NaCl at a flow rate of 32 mL/h. After 24 hours, the retentate was isolated containing approximately 96% of the original CMP-KDN. Quantification of CMP-KDN preparations were determined using the molar extinction coefficient of CMP (ε260=7,400). Purified and desalted sample aliquots were then freeze dried.

### Synthesis of CMP-3,7-Dideoxy-7-Azido-D-Glycero-D-Galacto-Nonulosonic Acid (CMP-Kdn7N₃)

KDN7N₃ (3,7-dideoxy-7-azido-D-glycero-D-galacto-nonulosonic acid) was enzymatically prepared using a Pasteurella multocida aldolase [24]. Typically, reactions contained 128 mM Tris pH 8.8, 17.5 mM 4-azido-4-deoxy-D-mannopyranose (Sussex Research Laboratories Inc.), 128 mM sodium pyruvate, and sufficient quantities of aldolase. Reactions were incubated at 37° C. for approximately 24 hours, and finally enzyme was removed by centrifugal ultrafiltration. Next, CMP-activation of synthesized KDN7N₃ was achieved enzymatically using a CMP-sialic acid synthetase from Campylobacter jejuni [25]. Here, reactions typically contained 50 mM Tris pH 9, 50 mM MgCl₂, 5 mM CTP, approximately 5 mM KDN7N₃, 4 units pyrophosphatase per mmole of CTP and approximately 0.68 mg/mL of CMP-sialic acid synthetase. Reactions were incubated at 37° C. for 2 hours, and finally enzyme was removed by centrifugal ultrafiltration. Filtered CMP-KDN7N₃ samples were then lyophilized and desalted/purified using a Superdex Peptide 10/300 GL (GE Healthcare) column with 10 mM ammonium bicarbonate. To achieve additional purity, elution fractions containing CMP-KDN7N₃ were subjected to anion-exchange chromatography (Mono Q 4.6/100 PE, GE Healthcare) using an ammonium bicarbonate gradient. Quantification of CMP-KDN7N₃ preparations were determined using the molar extinction coefficient of CMP (6260=7,400). Prior to lyophilization, NaCl was added to CMP-KDN7N₃ preparations in a molar ratio of 2:1 (salt: NulO).

For structural characterization of CMP-KDN and CMP-KDN7N₃, purified material was exchanged into 100% D₂O. Structural analysis was performed using either a Varian Inova 500 MHz (¹H) spectrometer with a Varian Z-gradient 3-mm probe or a Varian 600 MHz (¹H) spectrometer with a Varian 5 mm Z-gradient probe. All spectra were referenced to an internal acetone standard (δ_{H} 2.225 ppm and δ_{C} 31.07 ppm). Results are shown in Table 2 (CMP-KDN) and Table 3 (CMP-KDN7N₃) below verifying the production of each compound.

CMP-KDN and CMP-KDN7N₃ prepared compounds were also characterized using mass spectrometry (MS) or CE-MS analysis. For CE-MS, mass spectra were acquired using an API3000 mass spectrometer (Applied Biosystems/Sciex, Concord, ON, Canada). CE was performed using a Prince CE system (Prince Technologies, Netherlands). CE separation was obtained on a 90 cm length of bare fused-silica capillary (365 μm OD×50 μm ID) with CE-MS coupling using a liquid sheath-flow interface and isopropanol:methanol (2:1) as the sheath liquid. An aqueous buffer comprising 30 mM morpholine (adjusted to pH9 with formic acid) was used for experiments in the negative-ion mode. Alternatively, mass spectra were acquired using a SQD2 (Waters, Milford, Mass.). Here, the spectra were collected in the negative ion mode and no separations were attempted. The buffer used was a mixture of 1:1 acetonitrile/water with 0.31 mg/mL of ammonium bicarbonate.

Results verifying the production of each compound are shown in Table 4 below, where observed m/z ions from MS analysis correspond accurately to the calculated masses.

### Bacterial Strains and Growth Conditions

A mutant of N. gonorrhoeae strain F62 [26] that lacked expression of lipooligosaccharide glycosyltransferase D (IgtD), called F62 ΔIgtD [27], was provided by Dr. Daniel C. Stein (University of Maryland). LgtD adds a GalNAc residue to the terminal Gal of the HepI lacto-N-neotetraose species [28]. Therefore, any extension of the HepI of N. gonorrhoeae F62 ΔIgtD is limited to the addition of a nonulosonic acid residue that is transferred from the CMP-nonulosonate added to growth media.

Generally, bacteria (F62 ΔIgtD) grown overnight on chocolate agar plates were suspended in gonococcal liquid media supplemented with IsoVitaleX [29] that contained specified concentrations of the CMP-nonulosonate. Bacteria were then incubated at 37° C. for the period specified in each experiment.

### Antibodies

Goat anti-human fH was used in flow cytometry assays to detect human fH binding to bacteria. mAb 3F11 (mouse IgM; provided by Dr. Michael A. Apicella, University of Iowa) binds to the unsialylated HepI lacto-N-neotetraose structure; sialylation of LOS results in decreased binding of mAb 3F11 [30]. FITC conjugated anti-mouse IgM and anti-goat IgG were from Sigma.

### Flow Cytometry or FACS Assays

Flow cytometry for fH and mAb 3F11 binding were conducted as described in the art [31] and in U.S. patent application Ser. No. 14/627,396.

### Animal Model Experiments

The mouse model experiments and statistical analysis were conducted as described in the art [32] and in U.S. patent application Ser. No. 14/627,396.

### Serum Bactericidal Assay

Serum bactericidal assays were performed as follows, similar to methods outlined in [32,33] and in U.S. patent application Ser. No. 14/627,396. Bacteria were harvested from an overnight culture on chocolate agar plates and ˜10⁵ CFU of Ng were grown in liquid media containing the concentrations of CMP-NulO as specified for each experiment. Bacteria were diluted in Morse A and ˜2000 CFU of Ng F62 ΔIgtD were incubated with NHS (concentration specified for each experiment). The final reaction volumes were maintained at 150 μL. Aliquots of 25 μL of reaction mixtures were plated onto chocolate agar in duplicate at the beginning of the assay (t₀) and again after incubation at 37° C. for 30 minutes (t₃₀). Survival was calculated as the number of viable colonies at t₃₀ relative to t₀.

Substitution of Neisseria gonorrhoeae lacto-N-neotetraose (LNnT) lipooligosaccharide (LOS) with Neu5Ac results in the ability of the bacterium to evade complement-mediated killing. Prior studies have shown that the addition of Neu5Ac to LNnT LOS decreases binding of specific IgG and enhances binding of factor H (fH), an inhibitor of the alternative pathway of complement. Previously, we have shown that several CMP-activated nonulosonate (NulO) analogs, such as CMP-Neu5Gc, CMP-Neu5Gc8Me, CMP-Neu5Ac9Ac, CMP-Neu5Ac9Az and CMP-Leg5Ac7Ac can serve as substrates for gonococcal LOS sialyltransferase (Lst) (U.S. patent application Ser. No. 14/627,396). From this collection of CMP-NulOs tested, only CMP-Neu5Gc was able to simulate the high-level serum resistance reported for CMP-Neu5Ac, as well as a high level of fH binding to bacteria. Importantly, Neu5Gc differs from Neu5Ac at carbon 5, where it contains an N-glycolyl moiety rather than an N-acetyl one. The remainder of nonulosonates from this collection differ from Neu5Ac/Neu5Gc at either carbon 8, at carbon 9 or at carbons 7 and 9. We found that these CMP-NulO analogs, with changes to carbon 8, carbon 9 or carbons 7 and 9 of the NulO, did not enhance factor H binding, nor did they afford N. gonorrhoeae cells a high level of serum resistance. So, it appeared carbons 7, 8 and 9 within the exocyclic moiety of nonulosonate sugars played a critical role in the avoidance of serum mediated killing by N. gonorrhoeae, as evidenced by enhanced serum-mediated killing with CMP-Neu5Gc8Me, CMP-Neu5Ac9Ac, CMP-Neu5Ac9Az and CMP-Leg5Ac7Ac that was not observed with either CMP-Neu5Ac or CMP-Neu5Gc only feeding controls. So, in contrast to the other carbon 7, 8 and 9 variations, N-glycolyl substitution at the carbon 5-position did not have any negative impact on fH binding or serum resistance. We therefore proposed the use of CMP-nonulosonate analogs, with variations at carbons 7, 8 and/or 9, as a novel therapeutic/preventative strategy against the global threat of multi-drug resistant gonorrhea.

Surprisingly, we now report a different CMP-NulO with changes to the carbon 5 position of the NulO that can be utilized by N. gonorrhoeae Lst, will affect fH binding of bacteria, and result in serum sensitivity. In addition, this CMP-NulO analog was also found to have some efficacy against the antibiotic resistant ‘superbug’ H041 in the BALB/c vaginal colonization model. This CMP-NulO is CMP-3-deoxy-D-glycero-D-galacto-nonulosonic acid or CMP-deaminated neuraminic acid, also known as CMP-KDN. Like Neu5Gc, KDN differs from Neu5Ac at carbon 5, but unlike Neu5Ac or Neu5Gc with N-acetyl or N-glycolyl groups at carbon 5, respectively, KDN has just a hydroxyl group at carbon 5. An experimental summary for the testing of CMP-KDN is described below.

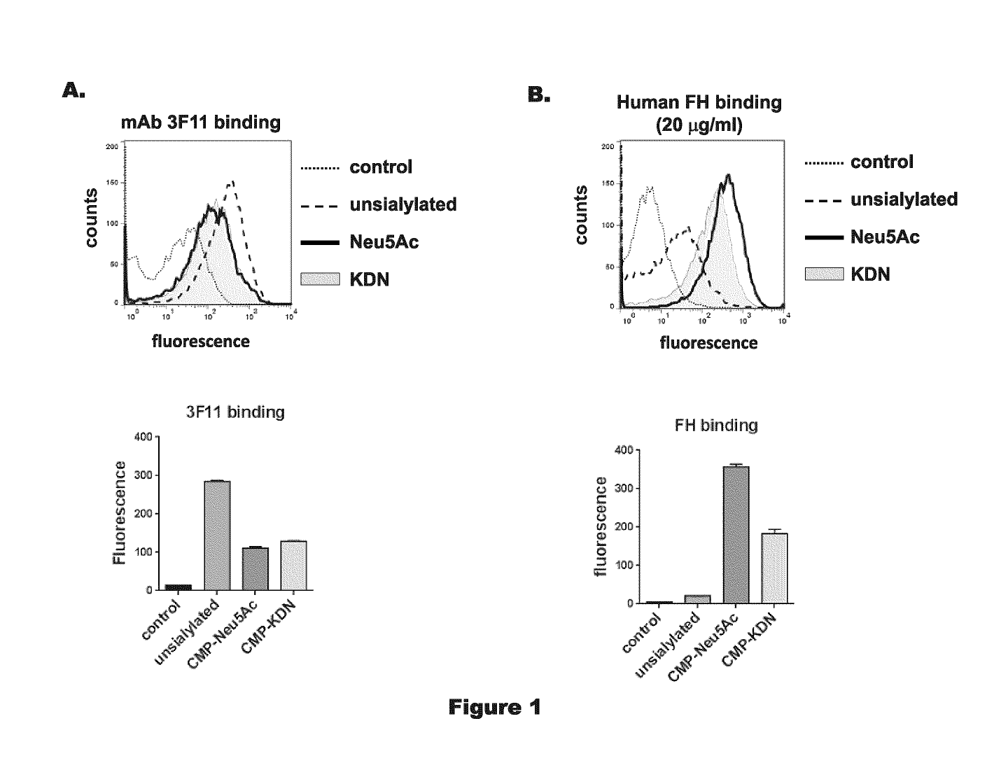

To determine if gonococcal LOS sialyltransferase (Lst) can utilize CMP-KDN, N. gonorrhoeae F62 ΔIgtD was grown in media alone (see ‘unsialylated’ in FIG. 1A), or media containing 20 μg/mL of either CMP-Neu5Ac or CMP-KDN (FIG. 1A), and bacteria were screened for binding of mAb 3F11 by flow cytometry. The mAb 3F11 binds to the terminal lactosamine residue of lacto-N-neotetraose (LNnT); any extension beyond the terminal Gal (in this instance, with a NulO) would abrogate 3F11 binding. As seen in FIG. 1A, growth in media containing CMP-KDN or CMP-Neu5Ac decreased binding of mAb 3F11 similarly, indicating that CMP-KDN served as a substrate for gonococcal Lst in the context of live bacteria and that KDN was incorporated onto LNnT. In addition, the ability of LNnT incorporated KDN to influence fH binding was examined (FIG. 1B). Here, fH binding to N. gonorrhoeae F62 ΔIgtD grown in the presence of 20 μg/mL of either CMP-Neu5Ac or CMP-KDN was examined by flow cytometry. Maximal fH binding was seen with Neu5Ac, whereas fH binding with KDN appeared to be half of that observed for Neu5Ac. This is in contrast to testing results with Leg5Ac7Ac, Neu5Ac9Az or Neu5Gc8Me nonulosonates, where they were found to not enhance fH binding above levels seen with an unsialylated F62 ΔIgtD control (U.S. patent application Ser. No. 14/627,396). So, KDN incorporation only modestly affects fH binding, and is further support for the incorporation of KDN within LNnT LOS. Based on these findings, KDN incorporation within N. gonorrhoeae LNnT LOS should not result in high serum sensitivity as that which is observed for Leg5Ac7Ac LNnT LOS incorporation, for example.

Addition of a terminal Neu5Ac residue to the LNnT LOS of N. gonorrhoeae that occurs in vivo or following the addition of CMP-Neu5Ac to growth media results in resistance to complement-dependent killing [34]. We next determined the effects of LNnT incorporation of KDN on the ability of N. gonorrhoeae F62 ΔIgtD to resist complement-dependent killing by normal human sera at concentrations of 3.3% or 10%. Bacteria were grown either in media alone, or media supplemented with 20 μg/mL of CMP-Neu5Ac or CMP-KDN. In addition, CMP-NulO competition experiments were also performed with CMP-Neu5Ac, CMP-Leg5Ac7Ac and CMP-KDN at CMP-NulO concentrations of either 20 μg/mL or 2 μg/mL as indicated, where the second CMP-NulO was added 15 minutes after the first (Table 1 below). To note, these CMP-NulO competition experiments are a method to examine the ability of select CMP-NulOs to counter the enhanced serum resistance due to CMP-Neu5Ac addition, providing information on their therapeutic potential (as any potential therapeutic will be required to compete with CMP-Neu5Ac in vivo). As shown in Table 1, CMP-Leg5Ac7Ac blocked serum resistance mediated by CMP-Neu5Ac at both 3.3% and 10% serum concentrations irrespective of the order of addition (ie CMP-Neu5Ac or CMP-Leg5Ac7Ac first). When CMP-KDN was examined alone, serum sensitivity was observed at 10% serum concentration, but complete serum resistance was observed at 3.3% serum concentration. In addition, CMP-KDN could only counter CMP-Neu5Ac induced serum resistance if it was provided before CMP-Neu5Ac, and again serum sensitivity was only observed with 10% serum concentrations. These results further suggest that the therapeutic potential of CMP-KDN should be lower than CMP-Leg5Ac7Ac.

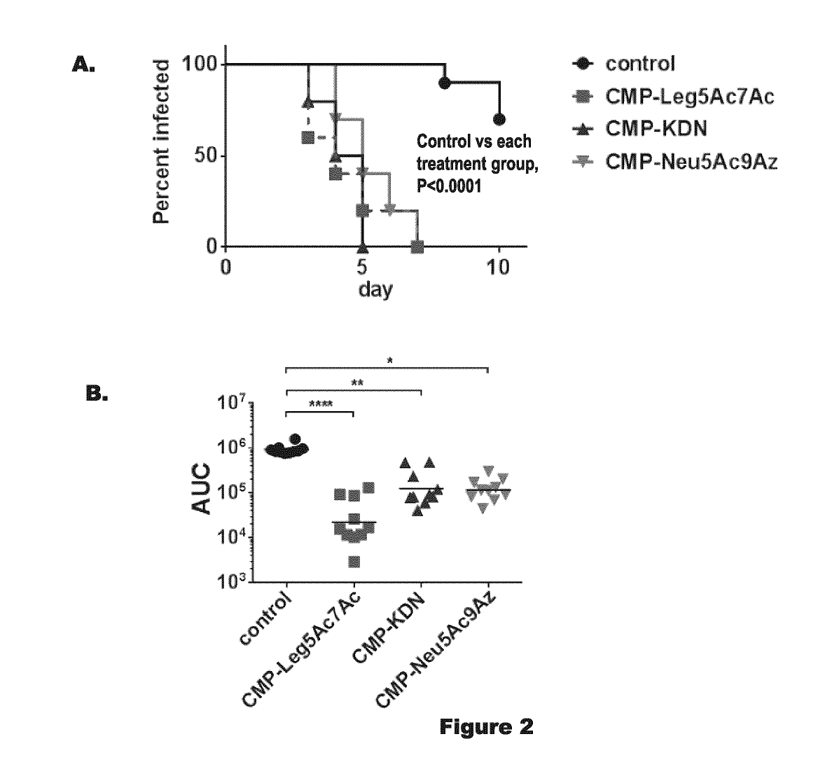

Regardless of the modest results obtained for CMP-KDN above, we still decided to pursue experiments evaluating the in vivo efficacy of this analog, somewhat due to the different phenotypes observed for KDN versus Neu5Gc, both NulOs with variation at carbon 5. The efficacy of CMP-KDN against the N. gonorrhoeae antibiotic resistant ‘superbug’ H041 was tested in the BALB/c mouse vaginal colonization model [35,32] (FIG. 2).

Four groups of Premarin treated BALB/c mice (10 mice per group) were infected as follows: i) H041→saline untreated control (‘control’), ii) H041→CMP-Leg5Ac7Ac (10 μg intravaginally daily), iii) H041→CMP-KDN (10 μg intravaginally daily), and iv) H041→CMP-Neu5Ac9Az (10 μg intravaginally daily). Treatment with all of these CMP-NulOs significantly attenuated N. gonorrhoeae H041 infection. Considering the ‘poor’ fH binding and bactericidal results obtained with CMP-KDN relative to CMP-Leg5Ac7Ac it is surprising that CMP-KDN is just as efficacious in an animal model of colonization.

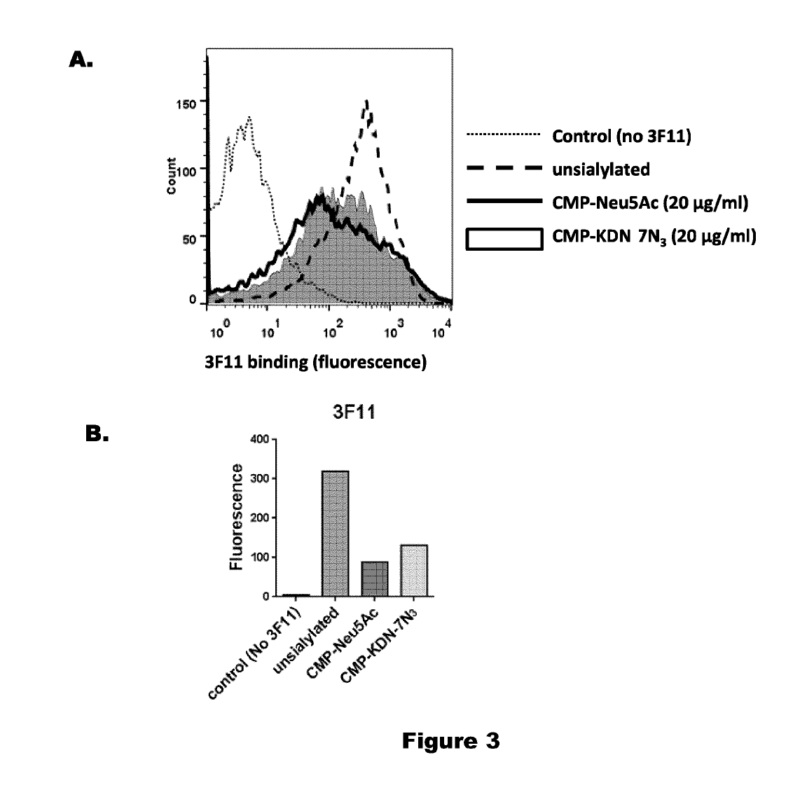

In efforts to test other CMP-KDN analogs, we chose to study CMP-KDN7N₃, differing from CMP-KDN only at the C7 position of the NulO. Similar to studies with CMP-KDN, we found CMP-KDN7N₃ could be utilized by gonococcal LOS sialyltransferase (Lst) (FIG. 3) using mAb 3F11 binding studies. In addition, we determined that addition of CMP-KDN7N₃ to growth media resulted in serum sensitivity of N. gonorrhoeae F62 ΔIgtD (albeit only at 10% serum concentrations) (Table 5), similar to results obtained with CMP-KDN (Table 1).

TABLE 1
Effect of CMP-KDN on complement killing of N. gonorrhoeae
F62 ΔlgtD. CMP-NulO concentrations are shown
in parentheses (μg/mL).

% survival in

| CMP-NulO added | 3.3% serum | 10% serum |
|---|---|---|
| None | 2 | 3 |
| CMP-Neu5Ac (20) alone | Not done | 115 |
| CMP-KDN (20) alone | 110 | 11 |
| CMP-Neu5Ac (20) | 107 | 108 |
| → ^{A} CMP-KDN (20) | | |
| CMP-KDN (20) | 107 | 11 |
| → ^{A} CMP-Neu5Ac (20) | | |
| CMP-KDN (20) | 107 | 10 |
| → ^{A} CMP-Neu5Ac (2) | | |
| CMP-Neu5Ac (20) | 9 | 5 |
| → ^{A} CMP-Leg5Ac7Ac (20) | | |
| CMP-Leg5Ac7Ac (20) | 7 | 3 |
| → ^{A} CMP-Neu5Ac (20) | | |
| CMP-Leg5Ac7Ac (20) | 6 | 6 |
| → ^{A} CMP-Neu5Ac (2) | | |

^{A} indicates 15 minutes interval before addition of next CMP-NulO.

TABLE 2
NMR chemical shifts δ (ppm) for CMP-3-deoxy-
D-glycero-D-galacto-nonulosonic acid (CMP-KDN).

| H3ax | H3ax | | |
|---|---|---|---|
| H3eq | 2.44 | C3 | 42.1 |
| H4 | 4.03 | C4 | 69.7 |
| H5 | 3.60 | C5 | 71.1 |
| H6 | 4.09 | C6 | 74.1 |
| H7 | 3.75 | C7 | 69.7 |
| H8 | 3.94 | C8 | 70.9 |
| H9 | 3.67; 3.92 | C9 | 64.3 |

TABLE 3
NMR chemical shifts δ (ppm) for CMP-3,7-dideoxy-
7-azido-D-glycero-D-galacto-nonulosonic acid (CMP-KDN7N₃).

| | | | |
|---|---|---|---|
| H3ax | 1.67 | | |
| H3eq | 2.48 | C3 | 42.0 |
| H4 | 4.03 | C4 | 69.7 |
| H5 | 3.61 | C5 | 72.2 |
| H6 | 4.20 | C6 | 73.8 |
| H7 | 3.82 | C7 | 62.3 |
| H8 | 4.06 | C8 | 69.7 |
| H9 | 3.77; 3.94 | C9 | 64.1 |

TABLE 4
MS data for CMP-3-deoxy-D-glycero-D-galacto-nonulosonic
acid (CMP-KDN) and CMP-3,7-dideoxy-7-azido-D-glycero-D-galacto-
nonulosonic acid (CMP-KDN7N₃).

| | Observed | Calculated | Formula | |
|---|---|---|---|---|
| Compound | m/z | mass | (M) | Comments |
| CMP-KDN | 572.4 | 573.4 | C₁₈H₂₈O₁₆N₃P | [M-H]⁻ |
| CMP-KDN7N₃ | 597.1 | 598.4 | C₁₈H₂₇O₁₅N₆P | [M-H]⁻ |

TABLE 5
Effect of CMP-KDN7N₃ on complement killing of
N. gonorrhoeae F62 ΔlgtD. CMP-NulO concentrations
are shown in parentheses (μg/mL).

% survival in

| CMP-NulO added | 3.3% serum | 10% serum |
|---|---|---|
| None | 17.47 | 3.40 |
| CMP-Neu5Ac (20) | 133.03 | 129.02 |
| CMP-KDN7N₃ (20) | 125.60 | 33.92 |
| CMP-KDN7N₃ (100) | 123.67 | 22.52 |

The scope of the claims should not be limited by the preferred embodiments set forth in the examples, but should be given the broadest interpretation consistent with the description as a whole.

The present description refers to a number of documents, the content of which is herein incorporated by reference in their entirety.

### REFERENCES

1. Chen, X., and Varki, A. (2010) ACS Chem. Biol. 5(2), 163-176.
2. Varki, A., and Gagneux, P. (2012) Ann. N Y Acad. Sci. 1253, 16-36.
3. Fearon, D. T. (1978) Proc. Natl. Acad. Sci. USA 75(4), 1971-1975.
4. Kajander, T., Lehtinen, M. J., Hyvarinen, S., Bhattacharjee, A., Leung, E., Isenman, D. E., Meri, S., Goldman, A., and Jokiranta, T. S. (2011) Proc. Natl. Acad. Sci. USA 108(7), 2897-2902.
5. Severi, E., Hood, D. W., and Thomas, G. H. (2007) Microbiology 153(Pt 9), 2817-2822.
6. Elkins, C., Carbonetti, N. H., Varela, V. A., Stirewalt, D., Klapper, D. G., and Sparling, P. F. (1992) Mol Microbiol 6(18), 2617-2628.
7. Ram, S., Sharma, A. K., Simpson, S. D., Gulati, S., McQuillen, D. P., Pangburn, M. K., and Rice, P. A. (1998) J. Exp. Med. 187(5), 743-752.
8. Wu, H., and Jerse, A. E. (2002) Sialylation of gonococcal LOS occurs during experimental murine gonococcal genital tract infection. In: Caugant, D. A., and Wedege, E. (eds). 13th International Pathogenic Neisseria Conference, Oslo, Norway.
9. Nakata, D., Munster, A-K., Gerardy-Schahn, R., Aoki, N., Matsuda, T., and Kitajima, K. (2001) Glycobiology 11: 685-692.
10. Inoue S. and Kitajima, K. (2006) Glycoconjugate Journal 23: 277-290.
11. Terada, T., Kitazume, S., Kitajima, K., Inoue, S., Ito, F., Troy, F. A., and Inoue, Y. (1993) J. Biol. Chem. 268: 2640-2648.
12. Terada, T., Kitajima, K., Inoue, S., Koppert, K., Brossmer, R., and Inoue, Y. (1996) Eur. J. Biochem. 236: 852-855.
13. Hao, J., Vann, W. F., Hinderlich, S., and Sundaramoorthy, M. (2006) Biochem. J. 397: 195-201.
14. Lawrence, S. M., Huddleston, K. A., Tomiya, N., Nguyen, N., Lee, Y. C., Vann, W. F., Coleman, T. A., and Betenbaugh, M. J. (2001) Glycoconjugate Journal 18: 205-213.
15. Lawrence, S. M., Huddleston, K. A., Pitts, L. R., Nguyen, N., Lee, Y. C., Vann, W. F., Coleman, T. A., and Betenbaugh, M. J. (2000) J. Biol. Chem. 275: 17869-17877.
16. Inoue, S., Lin, S-L., Chang, T., Wu, S-H., Yao, C-W., Chu, T-Y., Troy II, F. A., and Inoue, Y. (1998) J. Biol. Chem. 273: 27199-27204.
17. Inoue, S., Kitajima, K., and Inoue, Y. (1996) J. Biol. Chem. 271: 24341-24344.
18. Go, S., Sato, C., Yin, J., Kannagi, R., and Kitajima, K. (2007) Biochem. Biophys. Res. Commun. 357: 537-542.
19. Yabu, M., Korekane, H., Hatano, K., Kaneda, Y., Nonomura, N., Sato, C., Kitajima, K., and Miyamoto, Y. (2013) Glycobiology 23: 634-642.
20. Angata, T., Matsuda, T., and Kitajima, K. (1998) Glycobiology 8: 277-284.
21. Khedri, Z., Muthana, M. M., Li, Y., Muthana, S. M., Yu, H., Cao, H., and Chen, X. (2012) Chem. Commun. 48: 3357-3359.
22. Chan, T-H. and Li C-J. (1992) J. Chem. Soc., Chem. Commun. 747-748.
23. Crich, D. and Navuluri, C. (2011) Org. Lett. 13: 6288-6291.
24. Li, Y.; Yu, H.; Cao, H.; Lau, K.; Muthana, S.; Tiwari, V. K.; Son, B.; Chen, X. (2008) Appl. Microbiol. Biotechnol. 79(6), 963-970.
25. Guerry, P.; Ewing, C. P.; Hickey, T. E.; Prendergast, M. M.; Moran, A. P. (2000) Infect. Immun. 68(12), 6656-6662.
26. Schneider, H., Griffiss, J. M., Williams, G. D., and Pier, G. B. (1982) J. Gen. Microbiol. 128(Pt 1), 13-22.
27. Song, W., Ma, L., Chen, R., and Stein, D. C. (2000) J. Exp. Med. 191(6), 949-960.
28. Yang, Q. L., and Gotschlich, E. C. (1996) J. Exp. Med. 183(1), 323-327.
29. McQuillen, D. P., Gulati, S., and Rice, P. A. (1994) Methods Enzymol. 236, 137-147.
30. Yamasaki, R., Nasholds, W., Schneider, H., and Apicella, M. A. (1991) Mol. Immunol. 28(11), 1233-1242.
31. Lewis L A, Vu D M, Vasudhev S, Shaughnessy J, Granoff D M, Ram S. MBio. 2013 Oct. 15; 4(5):e00339-13. doi: 10.1128/mBio.00339-13.
32. Gulati S, Zheng B, Reed G W, Su X, Cox A D, St Michael F, Stupak J, Lewis L A, Ram S, Rice P A. PLoS Pathog. 2013; 9(8):e1003559. doi: 10.1371/journal.ppat.1003559. Epub 2013 Aug. 29.
33. McQuillen D P, Gulati S, Rice P A. Methods Enzymol. 1994; 236:137-47.
34. Smith, H., Cole, J. A., and Parsons, N. J. (1992) FEMS Micobiol. Lett. 79(1-3), 287-292.
35. Jerse A. E., Wu, H., Packiam, M., Vonck, R. A., Begum, A. A., and Garvin, L. E. Front Microbial. 2011; 2:107.
36. Campanero-Rhodes, M. A., Solis, D., Carrera, E., de la Cruz, M. J., and Diaz-Maurino, T. (1999) Glycobiology 9: 527-532.
37. Go, S., Sato, C., Furuhata, K., and Kitajima, K. (2006) Glycocon. J. 23: 411-421.
38. Haselhorst, T., Munster-Kuhnel, A. K., Stolz, A., Oschlies, M., Tiralongo, J., Kitajima, K., Gerardy-Schahn, R., and von Itzstein, M. (2005) Biochem. Biophys. Res. Commun. 327: 565-570.
39. Hayakawa, T. and Varki A. (2012) Chapter 8 Human-specific changes in sialic acid biology. Post-Genome Biology of Primates, primatology Monographs, H. Hirai et al. (eds.) Springer.
40. Khedri, Z., Li, Y., Muthana, S., Muthana, M. M., Hsiao, C-W., Yu, H., and Chen, X. (2014) Carbohydrate Research 389: 100-111.
41. Padler-Karavani, V., Song, X., Yu, H., Hurtado-Ziola, N., Huang, S., Muthana, S., Chokhawala, H. A., Cheng, J., Verhagen, A., Langereis, M. A., Kleene, R., Schachner, M., de Groot, R. J., Lasanajak, Y., Matsuda, H., Schwab, R., Chen, X., Smith, D. F., Cummings, R. D., and Varki, A. (2012) J. Biol. Chem. 287: 22593-22608.
42. Schaper, W., Bentrop, J., Ustinova, J., Blume, L., Kats, E., Tiralongo, J., Weinhold, B., Bastmeyer, M., and Munster-Kuhnel, A-K. (2012) J. Biol. Chem. 287: 13239-13248.
43. Song, X., Yu, H., Chen, X., Lasanajak, Y., Tappert, M. M., Air, G. M., Tiwari, V. K., Cao, H., Chokhawala, H. A., Zheng, H., Cummings, R. D., and Smith, D. F. (2011) J. Biol. Chem. 286: 31610-31622.
44. Yu, S., Kojima, N., Hakomori, S-I., Kudo, S., Inoue, S., and Inoue Y. (2002) Proc. Nat. Acad. Sci. USA 99: 2854-2859.
45. Zanetta, J-P., Pons, A., Iwersen, M., Mariller, C., Leroy, Y., Timmerman, P., and Schauer, R. (2001) Glycobiology 11: 663-676.
46. Gulati et al. PLOS Pathogens doi:10.1371/journal.ppat.1005290, Dec. 2, 2015.

1. A method for treating or preventing a Neisseria gonorrhoeae infection in a subject, comprising administering to the subject an effective amount of a compound of general formula I below or a pharmaceutical composition comprising said compound, or a derivative thereof, or a pharmaceutically acceptable salt thereof, or a solvate or hydrate thereof, or a stereoisomer thereof 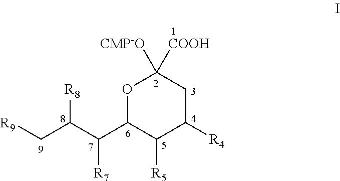
wherein:
R₅ is selected from the group consisting of: XR wherein X is O or S and R is H or a C₁ to C₆ linear, branched, saturated or unsaturated alkyl or cycloalkyl; NR′R″ wherein R′ and R″ are each independently H, a C₁ to C₆ linear, branched, saturated or unsaturated alkyl or cycloalkyl, or a substituted or unsubstituted phenyl or alkyl phenyl, or R′ and R″ together with N form a 5- or 6-member ring, optionally the ring is substituted with a C₁ to C₃ alkyl; XCYR wherein X and Y are each independently O or S and R is a C₁ to C₆ linear, branched, saturated or unsaturated alkyl or cycloalkyl or R is a substituted or unsubstituted phenyl or alkyl phenyl; and a halogen atom which is F, Cl, Br or I; and
R₄ and R₇ to R₉ are each independently selected from the group consisting of: H;
XR wherein X is O or S and R is H or a C₁ to C₆ linear, branched, saturated or unsaturated alkyl or cycloalkyl; OR′R″ wherein R′ and R″ are each independently H or a C₁ to C₆ linear, branched, saturated or unsaturated alkyl or cycloalkyl;
XCYR wherein X and Y are each independently O or S and R is a C₁ to C₆ linear, branched, saturated or unsaturated alkyl or cycloalkyl or R is a substituted or unsubstituted phenyl or alkyl phenyl; NR′R″ wherein R′ and R″ are each independently H, a C₁ to C₆ linear, branched, saturated or unsaturated alkyl or cycloalkyl, or R′ and R″ together with N form a 5- or 6-member ring, optionally the ring is substituted with a C₁ to C₃ alkyl; NH-acetyl; NH-thio-acetyl; NH-azido-acetyl; NH-(D-alanyl); NH-(N-acetyl-D-alanyl); N₃; O-Sia; O-Glc; benzamido [NHCOPh]; NH-Gly; NH-Succ; hexanoylamido [NHCO(CH₂)₄CH₃]; O-lactyl; O-phosphate; O-sulfate; and a halogen atom which is F, Cl, Br or I.

2. The method of claim 1, wherein:
R₄ is OH, O-acetyl, O-methyl, or NH₂;
R₅ is OH, O-acetyl, O-methyl, or sulfhydryl;
R₇ is OH, NH₂, O-acetyl, O-methyl, NH-acetyl, NH-azido-acetyl, NH-(D-alanyl), NH-(N-acetyl-D-alanyl), F, H, or N₃;
R₅ is OH, NH₂, N₃, O-acetyl, O-methyl, O-sulfate, O-Sia, or O-Glc; and
R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.

3. The method of claim 1, wherein the compound is of general formula IA below 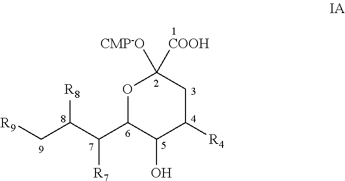
wherein R₄ and R₇ to R₉ are each independently as defined in claim 1.

4. The method of claim 1, wherein the compound is of general formula II below 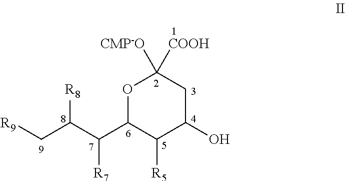
wherein R₅ and R₇ to R₉ are each independently as defined in claim 1.

5. The method of claim 1, wherein the compound is of general formula II below 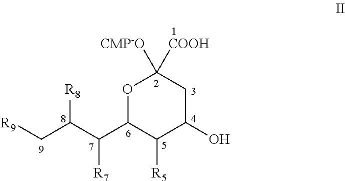
wherein:
R₅ is OH, O-acetyl, O-methyl, or sulfhydryl;
R₇ is OH, NH₂, O-acetyl, O-methyl, NH-acetyl, NH-azido-acetyl, NH-(D-alanyl), NH-(N-acetyl-D-alanyl), F, H, or N₃;
R₈ is OH, NH₂, N₃, O-acetyl, O-methyl, O-sulfate, O-Sia, or O-Glc; and
R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.

6. The method of claim 1, wherein the compound is of general formula II below 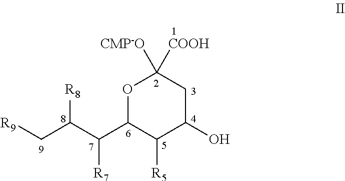
wherein:
R₅ is OH, F, Cl, Br, methyl, O-acetyl, O-methyl, or sulfhydryl;
R₇ OH, NH₂, O-acetyl, O-methyl, NH-acetyl, NH-azido-acetyl, NH-(D-alanyl), NH-(N-acetyl-D-alanyl), F, H, or N₃;
R₈ OH, NH₂, N₃, O-acetyl, O-methyl, O-sulfate, O-Sia, or O-Glc; and
R₉ OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.

7. The method of claim 1, wherein the compound is of general formula IIA below 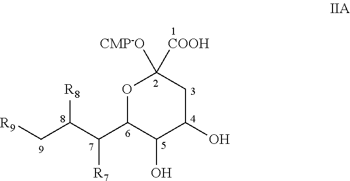
wherein R₇ to R₉ are each independently as defined in claim 1.

8. The method of claim 1, wherein the compound is of general formula IIA below 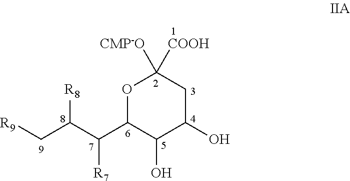
wherein:
R₇ is OH, NH₂, O-acetyl, O-methyl, NH-acetyl, NH-azido-acetyl, NH-(D-alanyl), NH-(N-acetyl-D-alanyl), F, H, or N₃;
R₈ is OH, NH₂, N₃, O-acetyl, O-methyl, O-sulfate, O-Sia, or O-Glc; and
R₉ is OH, O-acetyl, N₃, NH₂, NH-acetyl, NH-thio-acetyl, benzamido [NHCOPh], NH-Gly, NH-Succ, SCH₃, SO₂CH₃, hexanoylamido [NHCO(CH₂)₄CH₃], O-methyl, O-lactyl, O-phosphate, O-sulfate, O-Sia, F or H.

9. The method of claim 1, wherein the compound is of general formula III below 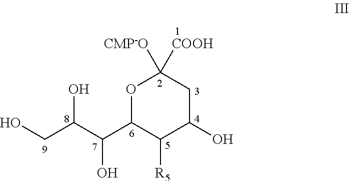
wherein R₅ is as defined in claim 1.

10. The method of claim 1, wherein the compound is of general formula IV below 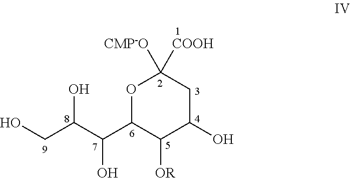
wherein R is H or a C₁ to C₆ linear, branched, saturated or unsaturated alkyl or cycloalkyl.

11. The method of claim 1, wherein the compound is compound V below

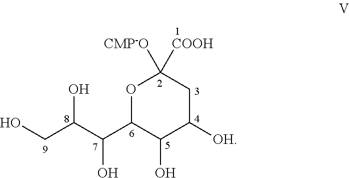

12. The method of claim 1, wherein the compound is cytidine 5′-monophospho-3-deoxy-D-glycero-D-galacto-nonulosonic acid (CMP-KDN) below

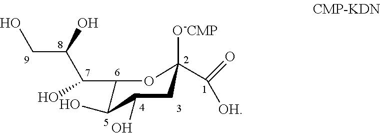

13. The method of claim 1, wherein the compound is of general formula VI below 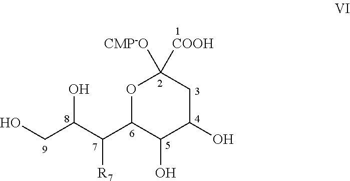
wherein R₇ is as defined in claim 1.

14. The method of claim 1, wherein the compound is of general formula VI below 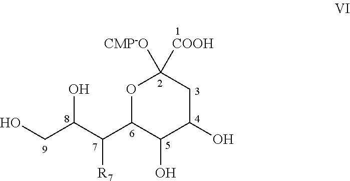
wherein R₇ is N₃, O-methyl, O-acetyl, NH₂ or a halogen atom.

15. The method of claim 1, wherein the compound is compound VII below

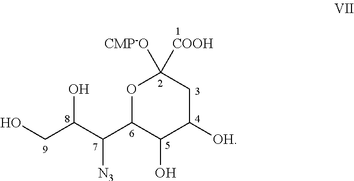

16. The method of claim 1, wherein the compound is cytidine 5′-monophospho-3,7-dideoxy-D-glycero-D-galacto-nonulosonic acid (CMP-KDN7N₃) below

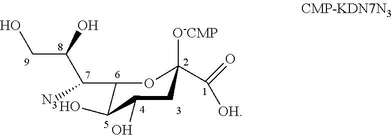

17. The method of claim 1, wherein the compound is:
3-deoxy-9-O-acetyl-D-glycero-D-galacto-nonulosonic acid (KDN9OAc) (R₄═OH, R₅═OH, R₇═OH, R₈═OH, R₉═O-acetyl);
3-deoxy-8-O-acetyl-D-glycero-D-galacto-nonulosonic acid (KDN8OAc) (R₄═OH, R₅═OH, R₇═OH, R₈═O-acetyl, R₉═OH);
3-deoxy-7-O-acetyl-D-glycero-D-galacto-nonulosonic acid (KDN7OAc) (R₄═OH, R₅═OH, R₇═O-acetyl, R₈═OH, R₉═OH);
3-deoxy-5-O-acetyl-D-glycero-D-galacto-nonulosonic acid (KDN5OAc) (R₄═OH, R₅═O-acetyl, R₇═OH, R₈═OH, R₉═OH);
3-deoxy-4-O-acetyl-D-glycero-D-galacto-nonulosonic acid (KDN4OAc) (R₄═O-acetyl, R₅═OH, R₇═OH, R₈═OH, R₉═OH);
3-deoxy-8,9-di-O-acetyl-D-glycero-D-galacto-nonulosonic acid (KDN8,9diOAc) (R₄═OH, R₅═OH, R₇═OH, R₈═O-acetyl, R₉═O-acetyl);
3-deoxy-9-O-methyl-D-glycero-D-galacto-nonulosonic acid (KDN9OMe) (R₄═OH, R₅═OH, R₇═OH, R₈═OH, R₉═O-methyl);
3-deoxy-8-O-methyl-D-glycero-D-galacto-nonulosonic acid (KDN8OMe) (R₄═OH, R₅═OH, R₇═OH, R₈═O-methyl, R₉═OH);
3-deoxy-7-O-methyl-D-glycero-D-galacto-nonulosonic acid (KDN7OMe) (R₄═OH, R₅═OH, R₇═O-methyl, R₈═OH, R₉═OH);
3-deoxy-5-O-methyl-D-glycero-D-galacto-nonulosonic acid (KDN5OMe) (R₄═OH, R₅═O-methyl, R₇═OH, R₈═OH, R₉═OH);
3-deoxy-4-O-methyl-D-glycero-D-galacto-nonulosonic acid (KDN4OMe) (R₄═O-methyl, R₅═OH, R₇═OH, R₈═OH, R₉═OH);
3-deoxy-8,9-di-O-methyl-D-glycero-D-galacto-nonulosonic acid (KDN8,9diOMe) (R₄═OH, R₅═OH, R₇═OH, R₈═O-methyl, R₉═O-methyl);
3,9-dideoxy-D-glycero-D-galacto-nonulosonic acid (9-deoxy-KDN) (R₄═OH, R₅═OH, R₇═OH, R₈═OH, R₉═H);
3,7-dideoxy-D-glycero-D-galacto-nonulosonic acid (7-deoxy-KDN) (R₄═OH, R₅═OH, R₇═H, R₈═OH, R₉═OH);
3,9-dideoxy-9-azido-D-glycero-D-galacto-nonulosonic acid (KDN9Az) (R₄═OH, R₅═OH, R₇═OH, R₈═OH, R₉═N₃);
3,9-dideoxy-9-fluoro-D-glycero-D-galacto-nonulosonic acid (KDN9F) (R₄═OH, R₅═OH, R₇═OH, R₈═OH, R₉═F); or
3,7-dideoxy-7-fluoro-D-glycero-D-galacto-nonulosonic acid (KDN7F) (R₄═OH, R₅═OH, R₇═F, R₈═OH, R₉═OH).

18. The method of claim 1, wherein the pharmaceutical composition comprises the compound and a pharmaceutically acceptable carrier; optionally the pharmaceutical composition comprises the compound and another therapeutic compound; optionally the therapeutic compound is selected from the group consisting of: other compounds used in the treatment or prevention of Neisseria gonorrhoeae infection, compounds used in the treatment or prevention of sexually transmitted diseases including Chlamidia trachomatis infection and HIV, and antibacterial peptides.

19.-20. (canceled)

21. The method of claim 1, wherein the subject is a mammal; optionally the subject is a human.

22.-25. (canceled)

26. A device coated or filled with a compound or a pharmaceutical composition as defined in claim 1.

27. (canceled)
